(19) United States
(12) Patent Application Publication
(10) Pub. No.: US 2013/0189725 A1
(43) Pub. Date: Jul. 25, 2013

(54) HUMAN IN VITRO MODEL OF THE BLOOD CEREBROSPINAL FLUID BARRIER

(71) Applicant: Horst Schroten, Langenfeld (DE)

(72) Inventor: Horst Schroten, Langenfeld (DE)

(73) Assignee: RUPRECHT-KARLS-UNIVERSITAT HEIDELBERG, Heidelberg (DE)

(21) Appl. No.: 13/817,181

(22) PCT Filed: Aug. 17, 2011
(86) PCT No.: PCT/EP2011/004149

§ 371 Date: Mar. 6, 2013

(30) Foreign Application Priority Data
Aug. 18, 2010 (GB) 1013829.5

(51) Int. Cl. C12Q 1/02

(52) U.S. Cl. CPC C12Q 1/025
USPC 435/29; 435/368

(57) ABSTRACT

The present invention relates to a human in vitro model system of the blood cerebrospinal fluid barrier (BCSFB), to a method for producing said model system, as well as to uses thereof.

The present invention relates to a human in vitro model system of the blood cerebrospinal fluid barrier (BCSFB), to a method for producing said model system, as well as to uses thereof.

In order to induce inflammation in the brain or elsewhere in the central nervous system (CNS), pathogens have to be able to pervade the blood brain barrier (BBB) made up of endothelial cells within CNS microvessels, as well as the BCSFB formed by the epithelial cells of the choroid plexus. Likewise, drugs that should be effective in the brain or in other parts of the CNS have to be able to pervade the BBB and the BCSFB.

Therefore, in order to study CNS-related pathogens and respective diseases, or to study and develop CNS-targeted drugs, it is important to provide easy-to-use but sophisticated and essentially complete in vitro model barrier systems which preferably allow for high-throughput experiments and mimic the physiological reality in animals and particularly in humans as accurately as possible. Such systems could be of high value for academic and industrial research, and could play an important role in the development of novel CNS-targeted drugs. In particular, the early assessment of the ability of a drug candidate to penetrate the CNS is critical and crucial during the drug development process.

The BCSFB regulates the exchange of cells and substances between the blood and the cerebrospinal fluid, and is formed by the epithelial cells of the choroid plexus. Dense connections between the cells which are set up by continuous strands of tight junctions (TJs) are required for barrier formation. TJs are molecular structures consisting of transmembrane proteins including claudins and occludin, as well as additional intracellularly associated membrane proteins like zonula occludens (ZO)-1 which connects the TJs to the actin cytoskeleton.

Further connections between the cells are mediated by the less tight adherence junctions (AJs), involving the transmembrane-protein E-cadherin.

Barrier-building cells display certain characteristics which enable them to restrict exchange across the cell layers to a minimum. In case of the endothelium representing the BBB, these cells are interconnected by a dense network of TJs and they exhibit a low pinocytotic activity concomitant with the absence of fenestrae. In the highly perfused choroid plexus, the endothelial cells are fenestrated and without TJs. Instead, the morphological correlate of the BCSFB is provided by an unique system of TJs between the cells of an outer epithelial layer. Properties of this cellular barrier are a high transepithelial electrical resistance (TEER), as well as a low permeability for macromolecules. During inflammatory events, these barriers undergo major alterations, leading to the opening of TJs, break-down of barrier function, and massive influx of cells of the immune system into the brain.

It has been traditionally believed that the role of the BCSFB in drug delivery to the CNS is not as significant as the role of the BBB. This belief was largely based on the fact that the surface area of the BCSFB is smaller than that of the BBB. However, this view has recently been challenged. In particular, it has been shown that the vascular perfusion of the choroid plexus is approximately eight times higher compared to that of the total brain. Further, the choroid plexus has a larger surface area than previously thought. Thus, the pharmaceutical industry has recently been undergoing a paradigm change and discontinues seeing the BBB as the one and only important factor for studying and investigating CNS-related physiological processes and events in the context of drug development.

Whereas today several human in vitro BBB models are well-established and validated, e.g. a model based on human brain microvascular endothelial cells (HBMEC), human in vitro systems displaying BCSFB functionality are not yet available. As a consequence, CNS-targeted drug candidates have only an 8% success rate and need a development time of 12 to 16 years, whereas non-CNS-targeted drug candidates have an 11% success rate and only 10 to 12 years development time. This discrepancy is due to the great complexity of the brain, side effects of centrally acting agents, low predictability of the existing animal models for humans, and the high selectivity of the BBB and BCSFB which prevent 98% of all CNS-targeted drug candidates from reaching their targets in the brain. Therefore, and in the absence of a “one-fits-it-all” solution, a variety of in vivo and in vitro models have to be used in parallel in order to obtain the best predictability of the clinical situation at an early stage. Thus, there exists an urgent need for the development of a human in vitro model of the BCSFB.

Further, the BCSFB is considered to play an important role in several brain- and/or CNS-related diseases. For example, despite the significant morbidity and mortality of bacterial meningitis, the pathogenesis of this disease in humans is still incompletely understood. Since the BBB and BCSFB are discussed as entry sites for the pathogens into the brain, an important factor for investigating this disease in the development of suitable in vitro systems mimicking the above barriers. However, respective in vitro model systems are limited to animal models, including rat cell lines and primary porcine choroid plexus epithelial cells. Therefore, also here an urgent need for the development of a human in vitro model of the BCSFB exists.

Accordingly, the technical problem underlying the present invention is to provide a functional human in vitro model of the BCSFB having a barrier functionality that reproduces the barrier functionality of the in vivo BCSFB to a large extent.

The solution to the above technical problem is achieved by the embodiments characterised in the claims.

In particular, the present invention relates to a model system that is based on human choroid plexus cells. By choosing adequate culture conditions, these cells display excellent BCSFB functionality, which is essentially conditioned and hallmarked by formation of continuous functional TJs by a dense grid of TJ proteins, a high TEER and only low macromolecule permeability. The human in vitro model system of the BCSFB of the present invention offers a wide range of applications in basic as well as industrial research concerning fields such as infectious diseases, neurology, oncology, psychiatry, pharmacology and drug development. For example, the model system of the present invention provides for the first time the possibility to study pathogens that exhibit their pathogenic activity exclusively in humans, such as meningococci. In particular, using the model system of the present invention, it could be shown that meningococci invade the human BCSFB substantially in a strictly polar manner, i.e. from the basolateral side of the BCSFB. Further, it could be shown that capsulated meningococci invade the human BCSFB significantly worse than mutants without capsules.

More specifically, in one aspect, the present invention relates to a human in vitro model system of the blood cerebrospinal fluid barrier (BCSFB), comprising human choroid plexus cells.

In a preferred embodiment of the model system of the present invention, the cells are selected from the group consisting of human choroid plexus carcinoma cells, human choroid plexus papilloma cells, and immortalised primary human choroid plexus cells. In a particularly preferred embodiment of the model system of the present invention, the cells are cells of the human choroid plexus papilloma cell line HIBCPP.

Human choroid plexus carcinoma cells, human choroid plexus papilloma cells, and immortalised primary human choroid plexus cells, as well as methods for obtaining these, are known to a person skilled in the art. Further, the human choroid plexus papilloma cell line HIBCPP is known to a person skilled in the art. Morphologically, HIBCPP cells are an epithelial cell arrangement with a monolayer system. They exhibit pleomorphic and neoplastic features, lack contact inhibition and can overlap each other. Herein, it is shown that HIBCPP, as well as human choroid plexus cells in general, display fundamental properties of a functional BCSFB in vitro. Most importantly, human choroid plexus cells present high TEER values when grown on transwell filter supports, which resemble the values observed in vivo in animal models. Additionally, human choroid plexus cells develop a low permeability for the paracellular flux of macromolecules. The development of a sufficiently high TEER concomitantly with a low permeability for macromolecules requires the formation of continuous functional TJs, which is a crucial feature of a valid BCSFB model. The presence of continuous TJs in the model system of the present invention has been clearly demonstrated by immunofluorescence.

Accordingly, in a preferred embodiment of the model system of the present invention, the human choroid plexus cells have been grown on the filter membrane of a transwell filter, i.e. the human choroid plexus cells are adherent to said filter membrane. Suitable transwell filters are known to a person skilled in the art. In particular, transwell filters comprise an upper well and a lower well, wherein the bottom of the upper well is formed by a filter membrane and the upper well fits into the lower well.

In a particularly preferred embodiment of the model system of the present invention, the human choroid plexus cells have been grown on the upper side of the filter membrane of a transwell filter, and are adherent thereto. Accordingly, the apical side of said cells, i.e. the side that is facing the CNS in vivo, is accessible from the upper well of the transwell filter. This setup is sometimes referred to herein as standard transwell filter system. In another particularly preferred embodiment of the model system of the present invention, the human choroid plexus cells have been grown on the lower side of the filter membrane of a transwell filter, and are adherent thereto. Accordingly, the basolateral side of said cells, i.e. the side that is facing the blood in vivo, is accessible from the upper well of the transwell filter. This setup is sometimes referred to herein as inverted transwell filter system.

In a preferred embodiment of the model system of the present invention, the human choroid plexus cells are characterised by formation of continuous functional TJs by a dense grid of tight junction proteins, a high TEER, and a low permeability for macromolecules.

Methods for determining whether a cell layer forms TJs are well known to a person skilled in the art, and include the analysis of the expression of TJ proteins, e.g. by reverse transcriptase polymerase chain reaction (RT-PCR), immunohistochemistry using antibodies against. TJ proteins, and electron microscopy. Methods for measuring the TEER of a cell layer are also known to a person skilled in the art, and include the use of an epithelial tissue voltohmmeter. Methods for determining a cell layer's permeability for macromolecules are known to a person skilled in the art as well, and include the determination of the passage of labelled tracer solutions from the apical to the basolateral side of a cell layer or vice versa.

The TEER of a cell layer is considered to be a “high TEER” as used herein, when the TEER is at least 250 Ω×cm², preferably at least 350 Ω×cm², and more preferably at least 450 Ω×cm². In a particularly preferred embodiment of the model system of the present invention, the human choroid plexus cells have a TEER of at least 250 Ω×cm².

The permeability for macromolecules of a cell layer is considered to be a “low permeability for macromolecules” as used herein, when the permeability is at most 2%, preferably at most 1%, and more preferably at most 0.5%.

In a further preferred embodiment of the model system of the present invention, the human choroid plexus cells are characterised by expression of claudin-1, claudin-2, claudin-3, ZO-1, occludin, E-cadherin, and transthyretin. Methods for determining the expression of specific proteins in cells are well known to a person skilled in the art.

The model system of the present invention is preferably obtainable by the method described hereinafter.

Thus, in a further aspect, the present invention relates to a method for producing a human in vitro model system of the BCSFB, comprising the step of culturing human choroid plexus cells on the filter membrane of a transwell filter. Suitable transwell filters are as defined above and are well known to a person skilled in the art. Methods for culturing cells on the filter membrane of transwell filters are also known to a person skilled in the art.

In a preferred embodiment of the method of the present invention, the cells are selected from the group consisting of human choroid plexus carcinoma cells, human choroid plexus papilloma cells, and immortalised primary human choroid plexus cells. In a particularly preferred embodiment of the method of the present invention, the cells are cells of the human choroid plexus papilloma cell line HIBCPP.

In a preferred embodiment of the method of the present invention, the human choroid plexus cells are cultured on the upper side of the filter membrane of the transwell filter, thus producing a standard transwell filter system as defined above. In another preferred embodiment of the method of the present invention, the human choroid plexus cells are cultured on the lower side of the filter membrane of the transwell filter, thus producing an inverted transwell filter system as defined above.

In a preferred embodiment of the method of the present invention, serum is withdrawn from the culture medium after the human choroid plexus cells reach confluence. The term “serum is withdrawn from the culture medium” as used herein relates to the fact that after they have grown to confluence, cells are further grown in medium containing no serum, or containing significantly less serum than during their growth to confluence. In particularly preferred embodiments of the method of the present invention, human choroid plexus cells are grown to confluence in medium containing 15% (v/v) serum, and are then further grown in medium containing no serum, or in medium containing 1% (v/v) serum.

In another aspect, the present invention relates to the use of human choroid plexus cells for the production of a human in vitro model system of the BCSFB. In a preferred embodiment thereof, the cells and the model system are as defined above.

In a further aspect, the present invention relates to the use of the human in vitro model system of the BCSFB according to the present invention for studying the human blood cerebrospinal fluid barrier (BCSFB).

In preferred embodiments of this aspect, studying the BCSFB includes the analysis/testing of drugs or drug candidates with respect to their ability to pass the BCSFB, as well as basic research that is related to the BCSFB in the fields of infection, multiple sclerosis, neurology, and oncology.

The figures show:

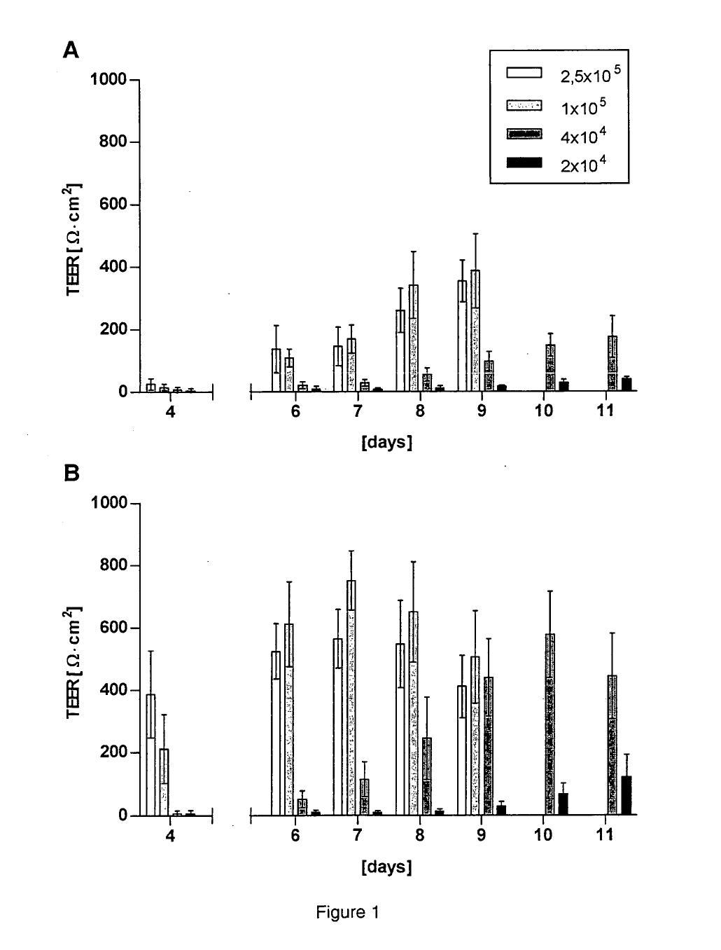

FIG. 1: HIBCPP develop high TEER in standard and inverted transwell filter systems. HIBCPP were seeded on transwell filters in the amounts indicated in the legend. Cells were grown either on the upper side (standard transwell filter system, A) or the lower side (inverted transwell filter system, B) of the filters. TEER was measured over time at the days after seeding of the cells as indicated on the x-axis. Shown is the mean+/−standard deviation (SD) of four (standard culture) or five (inverted culture) experiments, respectively, each performed in triplicates.

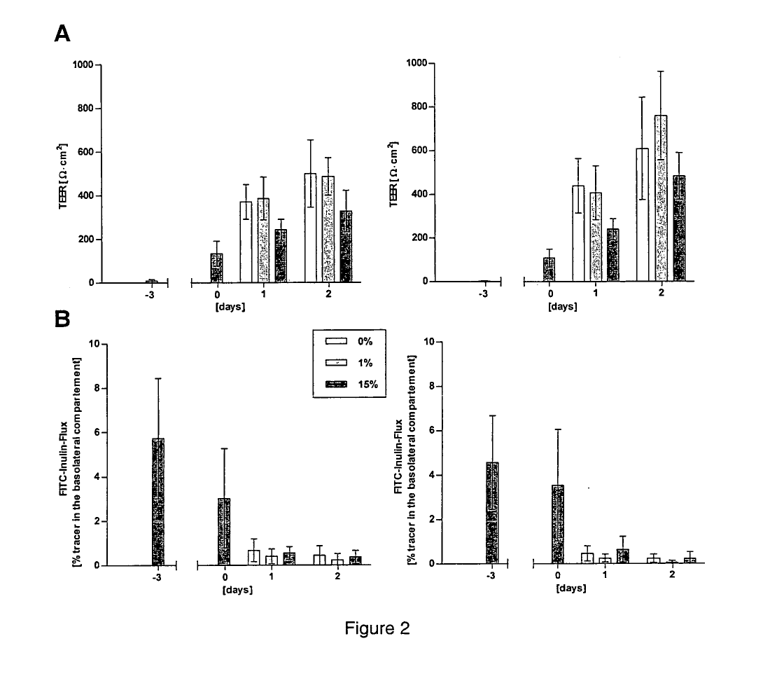

FIG. 2: High TEER values correlate with low FITC-inulin flux through HIBCPP-layers. HIBCPP were grown until a TEER above 70 Ω×cm² was measured (day 0) and subsequently cultured in 15%, 1%, or no serum, respectively, as indicated. At the indicated days, TEER (A) and the FITC-inulin flux (B) were determined. Cells were grown in the standard transwell filter system (5×10⁵ cells; left panels) or the inverted transwell filter system (4×10⁴ cells; right panels). Shown is the mean+/−SD of eight experiments performed in triplicates.

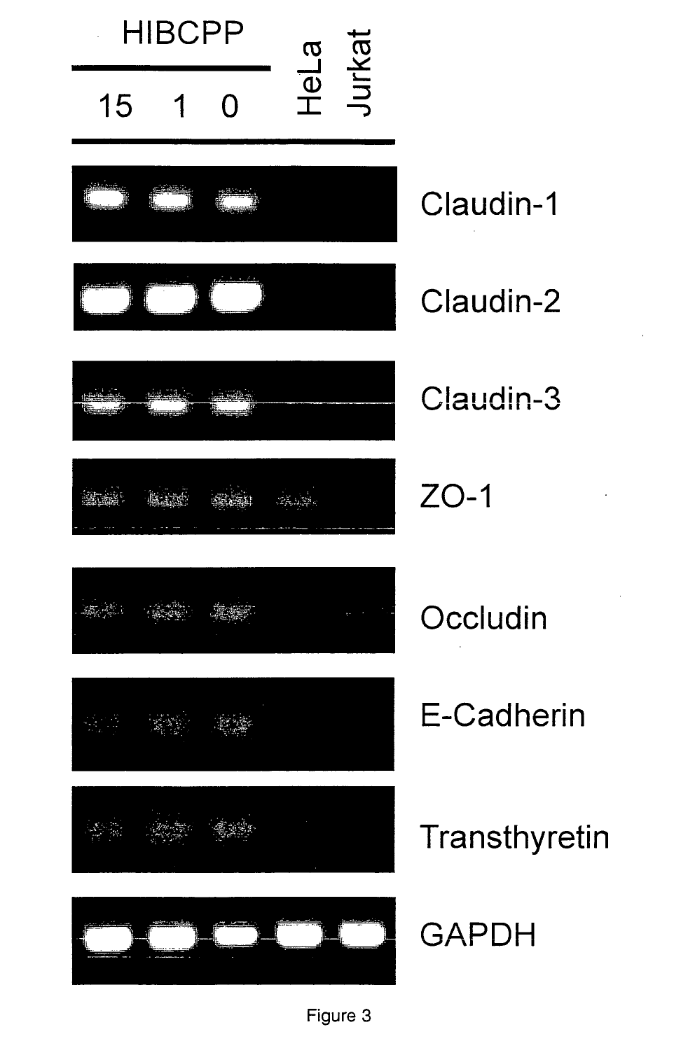

FIG. 3: RT-PCR analysis of the expression of junctional proteins and of transthyretin in HIBCPP. HIBCPP were grown in 6-well plates until confluence and subsequently cultured for 1 day in medium containing 15, 1 or 0% serum as indicated at the top of the lanes. The expression of the genes indicated at the right was analysed by RT-PCR. For comparison, RNA isolated from HeLa and Jurkat cells was analysed as well. Expression of the GAPDH gene served as control. The results shown are a typical example from three independently performed experiments.

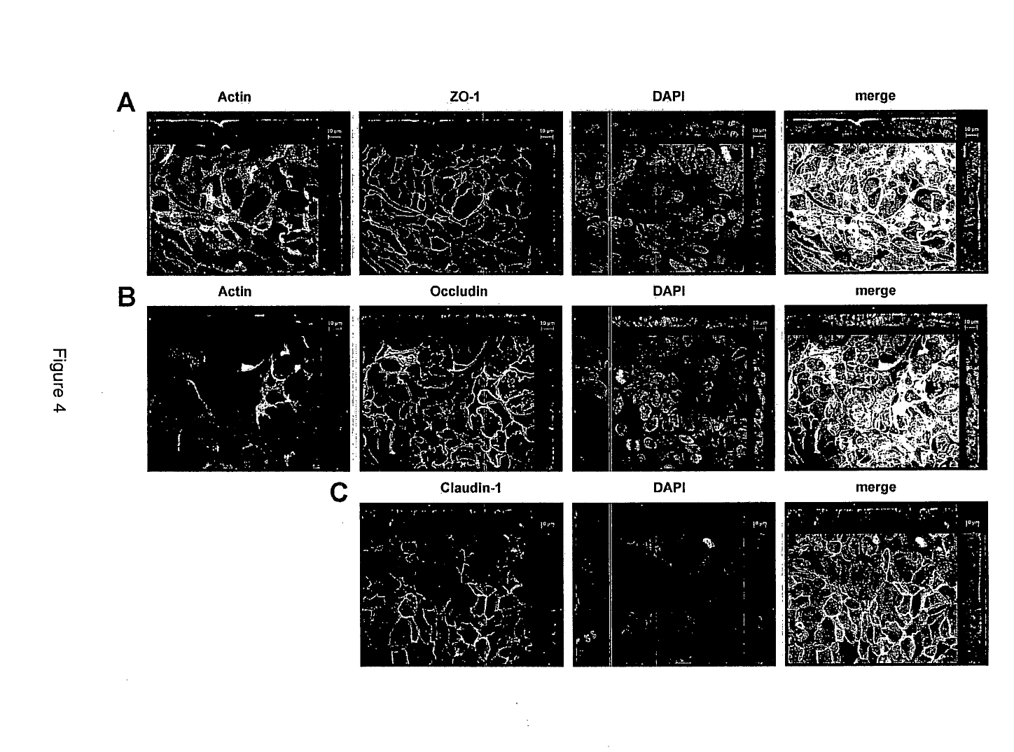

FIG. 4: HIBCPP display continuous tight junction strands. HIBCPP were stained for detection of ZO-1 (A), Occludin (B) and Claudin-1 (C). Pictures presented are Apotome-generated images; bottom of each panel is an xy en face view of a cell culture monolayer shown in a maximum-intensity projection through the z-axis; top and side of each panel is a cross section through the z-plane of multiple optical slices. In A and B, the actin cytoskeleton was stained with phalloidin-FITC in parallel. Since Claudin-1 samples were fixed with methanol, a qualitatively sufficient actin staining could not be observed. In all samples nuclei were stained with DAPI. The images shown are representative example of multiple stainings.

The present invention will now be further illustrated in the following examples without being limited thereto.

### EXAMPLES

### Experimental Procedures

Cultivation of HIBCPP on Transwell Filters.

HIBCPP were cultured in a 1:1 mixture of DMEM medium and HAM's F12 medium, supplemented with 4 mM L-Glutamine, 5 μg/ml insulin, penicillin (100 U/ml) and streptomycin (100 μg/ml), and 15% (v/v) heat inactivated fetal calf serum (FCS) [HIBCPP-medium with 15% (v/v) FCS]. Since HIBCPP have been described to change doubling time with increasing passages, only cells between passage 33 and 37 were used. For filter-based assays, the amounts of cells indicated in the respective experiments were seeded on the filter membrane of transwell filters (pore diameter 3.0 μm, pore density 2.0×10⁶ pores per cm², 0.33 cm²; Greiner Bio-one, Frickenhausen, Germany). For the standard transwell filter system, cells were seeded into the upper well of the transwell filter. Subsequently, cells were washed once each of the following two days. Medium was added to the lower well not before day two after seeding. For the inverted transwell filter system, the cells were seeded on the filter membrane of transwell filters that were flipped over and placed in a medium-flooded 12-well plate. Cells were fed the following day and the filters were flipped over again on day 2 after seeding. Upon confluence, HIBCPP had a seeding density of approximately 1.25×10⁶ cells per cm² (evaluated by 4,6-diamidino-2-phenylindole staining of the cell nuclei using immunofluorescence imaging). When TEER values became greater than 600 Ω×cm², cell culture was continued in HIBCPP-medium containing 15% (v/v), 1% (v/v) or no serum as indicated in the respective experiments. Cells were used in the experiments 1 or 2 days later when the TEER became greater than 250 Ω×cm², but were below 800 Ω×cm².

Measurement of TEER.

The TEER was measured using an epithelial tissue voltohmmeter (Millipore, Schwalbach, Germany) according to the manufacturer's instructions.

Determination of Paracellular Permeability.

Paracellular permeability of HIBCPP monolayers was determined by measuring the passage of a FITC-inulin (Sigma, Deisenhofen, Germany) tracer solution (100 μg/ml) from the apical to the basolateral compartment of transwell filters carrying the monolayers in a Tecan Infinite M200 Multiwell reader (Tecan, Switzerland) employing Magellan V6.6 software.

Immunohistochemistry.

Immunohistochemistry was performed as follows. HIBCPP were plated and observed in transwell filters. For phalloidin staining of the actin cytoskeleton as well as tight junction staining of ZO-1 and occludin, HIBCPP were fixed with 4% formaldehyde (w/v in PBS) at room temperature for 10 min, whereas for claudin-1 staining HIBCPP were fixed with ice-cold methanol for 20 min and thereafter permeabilised by applying 0.5% Triton X-100/1% BSA (v/v in PBS). Immunofluorescence staining was performed using primary antibodies (polyclonal rabbit anti-ZO-1, anti-occludin, or anti-claudin-1 at 1:250 dilution over night) obtained from Zymed (San Francisco, USA) and fluorophor-labelled secondary antibodies (polyclonal chicken anti-rabbit-IgG AlexaFluor 594 at 1:250 dilution for 60 min at 4° C.) obtained from Molecular Probes (Oregon, USA). Actin was stained by incubating the cells with phalloidin AlexaFluor 488 (1 U/300 μl; Molecular Probes, Oregon, USA) for 60 min at 4° C. Nuclei were stained with 4′-6-diamidino-2-phenylindole dihydrochloride (DAPI) (1:50000). Images were acquired with a Zeiss Apotome microscope and Axiovision software (Carl Zeiss, Jena, Germany) using a 63×/1.4 objective lens. This system provided an optical slice view reconstructed from fluorescent samples. FIG. 4 shows a representative selection of images that have been chosen from multiple standard microscopic fields. All immunofluorescence experiments were performed on Transwell filters in duplicate for each value and repeated at least three times.

RT-PCR.

Total cellular RNA was isolated using the RNeasy mini kit (Qiagen, Hilden, Germany) and subsequently treated with RNase-free DNase I (Roche, Grenzach-Wyhlen, Germany). After spectrophotometrical determination of the RNA concentration, 1 μg of total RNA was reverse transcribed with the Affinity Script QPCR cDNA synthesis kit according to the instructions provided by the manufacturer (Stratagene, La Jolla, Calif.). The following PCR reactions were performed with the Tag PCR core kit (Qiagen, Hilden, Germany) applying 0.5 μl of the generated cDNA again following the instructions provided by the manufacturer. PCR reactions mixtures were heated to 94° C. for 2 min and then subjected to 35 cycles of denaturation (94° C., 30 sec), annealing (60° C., 30 sec) and extension (72° C., 2 min) followed by a final extension step at 72° C. for 7 min. Subsequently, PCR products were visualised by agarose gel electrophoresis and ethidium bromide staining. Primers employed for PCR amplification were 5′-GCCAAGCAATGGCAGTCTC-3′ (SEQ ID NO: 1) and 5′-CTGGGCCGAAGAAATCCCATC-3′ (SEQ ID NO: 2) for ZO-1,5′-AACACCATT ATCCGGGACTTCT-3′ (SEQ ID NO: 3) and 5′-CGCGGAGTAGACGACCTTG-3′ (SEQ ID NO: 4) for claudin-3,5′-ATCCAAGTGTCCTCTGATGGT-3′ (SEQ ID NO: 5) and 5′-GCCAAGTGCCTTCCAGTAAGA-3′ (SEQ ID NO: 6) for transthyretin, 5′-GTTCGACAGTCAGCCGCATC-3′ (SEQ ID NO: 7) and 5′-GGA ATTTGCCATGGGTGGA-3′ (SEQ ID NO: 8) for the house keeping gene glyceraldehyde-3-phosphate dehydrogenase (GAPDH), 5′-AGGAACACATTT ATGATGAGCAG-3′ (SEQ ID NO: 9) and 5′-GAAGTCATCCACAGGCGAA-3′ (SEQ ID NO: 10) for occludin, 5′-GAAGATGAGGATGGCTGTCA-3′ (SEQ ID NO: 11) and 5′-AAATTCGTACCTGGCATTGA-3′ (SEQ ID NO: 12) for claudin-1, 5′-CCTGCCAATCCCGATGA-3′ (SEQ ID NO: 13) and 5′-TGCCCCATTCGT TCAAGTA-3′ (SEQ ID NO: 14) for E-cadherin, and 5′-ACCATTCCTTGACGG TGTCTA-3′ (SEQ ID NO: 15) and 5′-GCTGATTTTCCATTACGCCT-3′ (SEQ ID NO: 16) for claudin-2.

Measurement of Cell Viability.

Vitality of the cells was measured using a Life/Dead assay (Molecular Probes, Göttingen, Germany) according to the manufacturer's instructions. The results were photodocumented by fluorescence microscopy.

### Example 1

### HIBCPP Develop a Barrier Function on Transwell Filters In Vitro

To investigate the applicability of HIBCPP as human model system for the BCSFB, HIBCPP were grown on the filter membrane of transwell filters employing the standard transwell filter system as well as the inverted transwell filter system and TEER values were determined over time. As can be seen in FIG. 1, the HIBCPP developed a high membrane potential under both culture conditions. The time-point a detectable TEER started to develop did in both cases depend on the amount of cells seeded at the beginning of the experiment. TEER values reached up to about 500 Ω×cm² in the standard transwell filter system (FIG. 1A) and up to about 800 Ω×cm² in the inverted transwell filter system (FIG. 1B). About 3 days after the cells reached a high TEER, a decline of the membrane potential could be observed (2.5×10⁵ and 1×10⁵ cells seeded in FIG. 1B).

### Example 2

### Serum Withdrawal Increases HIBCPP Barrier Function

To elucidate whether serum withdrawal after confluence can lead to enhanced barrier properties including higher TEER values, HIBCPP were grown in the standard as well as the inverted transwell filter system until a TEER above 70 Ω×cm² was measured (designated day 0 in FIG. 2). Subsequently, the cells were cultivated in medium containing either 15%, 1% or no serum, respectively. Both in the standard and inverted transwell filter system HIBCPP reached higher TEER values when cell culture was continued in 1% or no serum following day 0 compared to continued growth in 15% serum (FIG. 2A).

### Example 3

### HIBCPP Develop Low Permeability for Macromolecules

A typical hallmark of the functional BCSFB is a low permeability for macromolecules. It was therefore investigated whether HIBCPP grown on transwell filters developed a low permeability for FITC-labelled inulin (FITC-inulin) concomitantly with the formation of a high TEER. As demonstrated in FIG. 2B, HIBCPP layers allow a high FITC-inulin flux (3% and higher) up to day 0 when TEER values are still low. Subsequently and simultaneously with the development of a high TEER, permeability for FITC-inulin dropped to levels below 1%. This decrease in macromolecular permeability can be observed with all three serum concentrations (15%, 1%, no serum) and can be detected in the standard as well as the inverted transwell filter system.

### Example 4

### Expression of TJ Proteins in HIBCPP

The polarisation of epithelial cells and the regulation of their barrier function is achieved by the expression of AJ and TJ proteins. To investigate if AJ and TJ components are present in HIBCPP, expression of a typical AJ protein (E-cadherin) and of several TJ associated factors (Claudin-1, -2, -3, ZO-1, Occludin) was determined by RT-PCR. As can be seen in FIG. 3, all investigated proteins can be found on RNA level in HIBCPP. Also Transthyretin, a typical marker protein for choroid plexus epithelial cells, was detected. Similar expression levels of all factors analysed were found when RNA isolated from HIBCPP cultured under different serum conditions (15%, 1% or no serum after confluence was reached) was analysed (FIG. 3).

### Example 5

### TJ Morphology of HIBCPP

To collect information concerning the TJ morphology of HIBCPP, HIBCPP layers grown in the inverted (FIG. 4) and standard (data not shown) transwell filter system were analysed by immunofluorescence against ZO-1 (FIG. 4A), occludin (FIG. 4B), and claudin-1 (FIG. 4C). Cells were grown until TEER values were above 70 Ω×cm² and subsequently cultivated for one more day in medium containing 1% serum. Cells prepared for immunofluorescence displayed a TEER above 250 Ω×cm², but were below 800 Ω×cm². Corresponding to the RT-PCR data, all three investigated proteins were expressed and detectable on protein level (FIG. 4). Importantly, the immunofluorescence signal of the three tight junction associated factors displayed a continuous pattern localised at the sites of cell-cell contact.

1. A human in vitro model system of the blood cerebrospinal fluid barrier (BCSFB), comprising human choroid plexus cells.

2. The human in vitro model system of the BCSFB according to claim 1, wherein said cells are selected from the group consisting of human choroid plexus carcinoma cells, human choroid plexus papilloma cells, and immortalised primary human choroid plexus cells.

3. The human in vitro model system of the BCSFB according to claim 2, wherein said cells are cells of the human choroid plexus papilloma cell line HIBCPP.

4. The human in vitro model system of the BCSFB according to claim 1, wherein said cells have been grown on the filter membrane of a transwell filter.

5. The human in vitro model system of the BCSFB according to claim 4, wherein said cells have been grown on the upper side of the filter membrane of the transwell filter and the apical side of said cells is accessible from the upper well of the transwell filter.

6. The human in vitro model system of the BCSFB according to claim 4, wherein said cells have been grown on the lower side of the filter membrane of the transwell filter and the basolateral side of said cells is accessible from the upper well of the transwell filter.

7. The human in vitro model system of the BCSFB according to claim 1, wherein said cells are characterised by formation of continuous functional tight junctions by a dense grid of tight junction proteins, a high transepithelial electrical resistance (TEER), and a low permeability for macromolecules.

8. The human in vitro model system of the BCSFB according to claim 1, wherein said cells have a TEER of at least 250 Ω×cm².

9. The human in vitro model system of the BCSFB according to claim 1, wherein said cells are characterised by expression of claudin-1, claudin-2, claudin-3, ZO-1, occludin, E-cadherin, and transthyretin.

10. A method for producing a human in vitro model system of the blood cerebrospinal fluid barrier (BCSFB), comprising the step of culturing human choroid plexus cells on the filter membrane of a transwell filter.

11. The method according to claim 10, wherein said cells are selected from the group consisting of human choroid plexus carcinoma cells, human choroid plexus papilloma cells, and immortalised primary human choroid plexus cells.

12. The method according to claim 11, wherein said cells are cells of the human choroid plexus papilloma cell line HIBCPP.

13. The method according to claim 10, wherein said cells are cultured on the upper side of said filter membrane.

14. The method according to claim 10, wherein said cells are cultured on the lower side of said filter membrane.

15. The method according to claim 10, wherein serum is withdrawn from the culture medium after said cells reach confluence.

16. The human in vitro model system of the BCSFB according to claim 1, which is obtainable by the method according to claim 10.

17. A method for analysing or testing the ability of a drug or drug candidate to pass the human blood cerebrospinal fluid barrier, said method comprising:
contacting said drug or drug candidate with a human in vitro model system of the blood cerebrospinal fluid barrier (BCSFB) comprising human choroid plexus cells; and
analysing or testing whether the drug or drug candidate passes the BCSFB.

18. The method according to claim 17, wherein said cells are selected from the group consisting of human choroid plexus carcinoma cells, human choroid plexus papilloma cells, and immortalised primary human choroid plexus cells.

19. The method according to claim 18, wherein said cells are cells of the human choroid plexus papilloma cell line HIBCPP.

20. (canceled)


SEQUENCE LISTING (16 sequences)

SEQ ID NO: 1 - DNA, 19 bp, Artificial Sequence
  Primer
gccaagcaatggcagtctc

SEQ ID NO: 2 - DNA, 21 bp, Artificial Sequence
  Primer
ctgggccgaagaaatcccatc

SEQ ID NO: 3 - DNA, 22 bp, Artificial Sequence
  Primer
aacaccattatccgggacttct

SEQ ID NO: 4 - DNA, 19 bp, Artificial Sequence
  Primer
cgcggagtagacgaccttg

SEQ ID NO: 5 - DNA, 21 bp, Artificial Sequence
  Primer
atccaagtgtcctctgatggt

SEQ ID NO: 6 - DNA, 21 bp, Artificial Sequence
  Primer
gccaagtgccttccagtaaga

SEQ ID NO: 7 - DNA, 20 bp, Artificial Sequence
  Primer
gttcgacagtcagccgcatc

SEQ ID NO: 8 - DNA, 19 bp, Artificial Sequence
  Primer
ggaatttgccatgggtgga

SEQ ID NO: 9 - DNA, 23 bp, Artificial Sequence
  Primer
aggaacacatttatgatgagcag

SEQ ID NO: 10 - DNA, 19 bp, Artificial Sequence
  Primer
gaagtcatccacaggcgaa

SEQ ID NO: 11 - DNA, 20 bp, Artificial Sequence
  Primer
gaagatgaggatggctgtca

SEQ ID NO: 12 - DNA, 20 bp, Artificial Sequence
  Primer
aaattcgtacctggcattga

SEQ ID NO: 13 - DNA, 17 bp, Artificial Sequence
  Primer
cctgccaatcccgatga

SEQ ID NO: 14 - DNA, 19 bp, Artificial Sequence
  Primer
tgccccattcgttcaagta

SEQ ID NO: 15 - DNA, 21 bp, Artificial Sequence
  Primer
accattccttgacggtgtcta

SEQ ID NO: 16 - DNA, 20 bp, Artificial Sequence
  Primer
gctgattttccattacgcct